(19) United States
(12) Patent Application Publication
(10) Pub. No.: US 2004/0076946 A1
(43) Pub. Date: Apr. 22, 2004

(54) Integrated barrel-mounted wine laboratory and winemaking apparatus

(76) Inventors: Kenneth B. Trauner, Belvedere, CA (US); Luiz B. Da Silva, Danville, CA (US); Alexander M. Rubenchik, Livermore, CA (US)

Correspondence Address: John P. Wooldridge, Esq., Suite 110, 535 Lipoa Parkway, Kihei, HI, 96753, US

(21) Appl. No.: 10/443,296

(22) Filed: May 22, 2003

Related U.S. Application Data
(63) Continuation-in-part of application No. 09/693,084, filed on Oct. 19, 2000.
(60) Provisional application No. 60/382,321, filed on May 22, 2002.

(51) Int. Cl. C12M 1/36; C12Q 3/00

(52) U.S. Cl. 435/3; 435/287.1; 435/286.1; 435/287.5

(57) ABSTRACT

A device is described that can be easily used to minimally invasively perform barrel related winemaking and wine testing tasks without need for movement of the barrel. System includes techniques for evaluating the condition and state of wine in the barrel. The device consists of a bung related device that connects to a sensor package, a laboratory system, fluid and chemical reservoirs and a remote monitoring and control station. The central station controls measurements and sampling of the wine and delivery of juice and chemicals to the barrel.

This application is a continuation-in-part of U.S. patent application Ser. No. 09/693,084, titled “Integrated Wine Quality Sensor”, filed Oct. 19, 2000 and incorporated herein by reference.

This application claims priority to U.S. Provisional Patent Application Serial No. 60/382,321, titled “Integrated Barrel-Mounted Wine Laboratory And Winemaking Apparatus”, filed May 22, 2002 and incorporated herein by reference.

### BACKGROUND OF THE INVENTION

1. Field of the Invention

The present invention relates to winemaking processes, and more specifically, it relates to the remote monitoring and control of the wine-making process during barrel aging.

2. Description of Related Art

Winemaking continues to use barrel-aging processes to allow the wines to mature and gain complex flavors. Deleterious processes are associated with barrel aging. Numerous processes are monitored by manually sampling the barrels. The sampling process is costly and labor intensive.

Monitoring is limited by the cost and physical effort associated with moving of the large and heavy barrels. Limitations in monitoring capabilities force the winemakers to track the performance of the wine aging process with infrequent sampling and testing. Infrequent testing increases the risk to winemakers by allowing adverse reactions in the barrels to go unobserved. Movement of the barrels for monitoring requires mechanical lowering of the barrels using mechanized hoists. The process is associated with adverse effects including agitation, spillage, and increased oxidation.

Cost and effort associated with manual testing prevents the winemakers from determining concentrations of key components in real time. Lack of adequate data on barrel performance, and evaporative losses over time prevents winemakers from optimizing use of the barrels. Lack of determination of variables prevents determination of wine maturation variables versus recent changes made in the wine parameters—additives, etc. Lack of knowledge of material and physical characteristics of the wine as it ages increases the guesswork. Failure to sample of all the barrels at frequent intervals leads to inaccuracies. Poorly behaving wine/barrels may be unobserved. Lack of sampling prevents determination of deleterious processes in specific barrels. Contamination with pathogens may be missed in individual barrels. Effort associated with moving barrels prevents control of the stirring and sedimentation processes.

Barrel aging varies with the type and varieties of wine produced. During the winemaking process, a number of variables are altered and monitored. Spot sampling of barrels is performed at monthly intervals for olfactory determination of off odors. Off odors leads to samples sent to regional laboratories. Wine compositions of numerous chemicals are determined, including but not limited to, sulfite concentration, degrees Brix, malic acid, lactic acid, pH, hydrogen sulfide, amino acids, and volatile acidity (acetic acid).

There presently exists a need to provide a minimally invasive technology that can allow winemakers real time monitoring and control of the winemaking process.

Numerous scientific techniques exist for monitoring the physical and chemical properties of fluids. Recent advances in microetectromechanical (MEMS) technologies, biosensors and microfluids allow for the development of complex laboratories on microscopic chip assemblies. Diagnostic systems for DNA and proteins have been incorporated into silicon chip and microscopic bead technologies for molecular, antigenic and proteonomic detection.

There exists a need for a simple and economical process controlling the barrel aging process. The present invention fulfills this need.

It is understood that the term “wine” also relates to any barrel aged fermenting beverage.

### SUMMARY OF THE INVENTION

It is an object of the present invention to provide a method and apparatus for measuring one or more parameters of a fermenting liquid and adjusting the contents of the fermenting liquid to change the measured parameter.

It is an object of the invention to provide a minimally invasive apparatus, mounted to the winemaking barrel and bung, for remotely monitoring physical, chemical and optical characteristics of the wine.

It is another object to provide the means to deliver juice, sulfite and other winemaking related chemicals accurately to the barrel without moving the barrel.

It is another object of the invention to provide a means to sense both the wine and wine vapor for undesirable aromatic compounds that may adversely affect the wine.

It is another object to monitor fluid levels within the barrel characterizing the evaporative losses and barrel performance.

It is an object of the invention to optimize the number of cycles that barrels are used by monitoring performance and degradation of performance over time. By eliminating poorly performing barrels and not discarding those continuing to perform well, this system also intends to reduce barrel-purchasing costs.

It is an object of the invention to vent the wine in the barrel.

It is another object of the invention to remotely monitor the behavior of the wine in the barrel and remotely control elements of the winemaking process.

It is another object of the invention to track the changes in wine behavior and to iteratively examine the data with artificial intelligence software for both advantageous and deleterious patterns to optimize winemaking algorithms for use in future wine production.

It is another object of the invention to remotely control the obtaining and sealing of aliquot wine samples from the barrel, without contamination, into sealed containers for shipment to offsite laboratories.

It is an object of the invention to measure one or more parameters of a fermenting liquid and electronically communicate the measurement to a computer or a user. The computer can automatically adjust the contents of the liquid. The user can manually make changes to the contents of the liquid. Radio frequency or other wireless means can be part of the communication means. Bluetooth technology can be part of the communication means.

These and other objects will be apparent to those skilled in the art based on the disclosure herein.

The present invention comprises a minimally invasive method for controlling and monitoring the production of wine in a barrel. An embodiment of the present method involves the use of a bung with numerous apertures with actively controlled valves to control the flow of juice into the barrel and the flow and sampling of gas from the barrel, a mixing arm attached to the bung for stirring the wine, a microscopic laboratory within the bung platform, a central processor and telemetry device for sending and receiving information from a central control computer, a reservoir and tubing to replenish juice and additives in the barrel. The stirring arm and bung contain numerous, physical, chemical and optical sensors and sampling devices. The method further provides for central computer comparison between barrels and batches of wine. Olfactory and chemical sensors will alert the controller to the presence of non-desirable aromatic compounds in the barrel.

The method further provides for an analysis of barrel performance by plotting the evaporative losses in real time and comparing performance to previous seasonal barrel performance, and control barrel performance.

The method also allows gas chromatography analysis and repeated comparison between barrels of the components within the fluid. Optionally, additional sampling of fluid properties of elements, such as electrical resistivity, scattering, temperature and optical density, may be tailored by the controller. Analysis of variables will also allow a geographic analysis of barrel performance within the winery to assure uniformity of the wine production.

The method includes a sulfite dispenser attached through a dedicated channel in the bung to allow for close control of sulfite concentrations in the wine especially during fermentation. The method also includes an actively controlled juice reservoir and pump system for replenishing juice in the barrels.

The method includes an active venting system through the bung with vented gases attached to tubing attached for sampling to a gas chromatography (GC) device. The method includes a system for flushing the tubing with inert gas prior to GC sampling and testing.

More specifically, one embodiment involves a method that comprises the steps of:

a) sampling the fluid for temperature, electrical resistivity, optical scattering, OD ratios at 480/580 nm, pH, sulfite concentration, Brix, volatile acidity, malic acid, lactic acid, ammonia, amino acids, transmission spectroscopy, electrical impedance, optical scattering;

b) at the control of the central computer—sampling fluid or gas vapor for complex analysis including gas chromatography or mass spectroscopy;

c) monitoring the fluid or vapor with olfactory sensors for nondesirable aromatic compounds;

d) measuring fluid levels and replenishing juice as needed. Analysing barrel evaporative rates as an indicator of barrel performance;

e) sampling SO₂ levels in the wine and administering sulfite to maintain levels as determined by the winemaker; and

f) monitoring scattering parameters of the wine, where a stirring device will gently agitate the fluid with rate and speed controlled by a microprocessor to achieve and maintain optical parameters determined by the winemaker to maintain desired amount of particulate suspension in the wine.

Advantageously, the method includes central computer controlled simultaneous data comparison between barrels of any desired parameters in real time.

The invention further provides a minimally invasive apparatus for sensing intrinsic properties of wine and wine vapor, treating wine with fluids and chemicals, stirring the wine, sensing of non-desirable aromatic compounds, monitoring wine and barrel performance over time and comparison between different barrels. One implementation of the apparatus is comprised of a bung unit containing multiple sensor systems, a wine stirring apparatus, multiple conduits for sampling fluid and vapor, and an active venting system;

a platform for securing the apparatus to the barrel;

a laboratory module with visual display;

a telemetry unit with a wireless transmitter/receiver;

a wine reservoir;

a sulfite container; and

a remote central computer.

The invention further provides an apparatus for monitoring fluid levels and administering juice to the barrel: One implementation of the apparatus is comprised of

electrical resistance sensor elements on the stirring rod;

a juice reservoir;

an actuator valve;

a pump; and

a microprocessor for sending and receiving the electrical resistance sensor data. The microprocessor controls the sensor and sends data to the central control computer and further controls the actuator valve and pump assembly to deliver juice to the barrel. Barrel evaporative performance is analyzed by the central computer. A usable fluid reservoir is of the type disclosed in U.S. Pat. No. 4,115,789, which is incorporated herein by reference.

The invention further provides an apparatus for continuous monitoring of the fluids for nondesirable aromatic compounds. One implementation of the apparatus is comprised of:

an olfactory sensor within the bung or stirring rod, where the sensor may be comprised of polymer coatings sensitive to aromatic compounds inducing color change on exposure to undesirable compounds or microscopic latex beads bound to antibodies to surface antigens associated with the nondesirable yeast or bacteria;

a scanning device to monitor for changes in the olfactory sensor;

a microprocessor for recording changes and transmitting to a central computer processor; and

a software based alert system.

The invention further provides an apparatus for continuous monitoring of the optical properties of the wine and an actively controlled stirring device for mixing the wine based on the optical data. One implementation of the apparatus comprises:

an optical sensor contained within a stirring rod to determine scattering and spectroscopic properties of the wine;

a spectrophotometer system including a light source and an optical fiber;

a central computer to analyze data and to control the operating parameters for the stirring apparatus;

a bung associated motor for manipulating the a stirring rod; and

a power source.

The invention further provides an apparatus for continuous monitoring of the fluids for hydrogen sulfide levels and administration of sulfite to the wine. The assembly comprises a reservoir driver and a powder delivery instrument. One implementation of the apparatus comprises:

a sulfide sensor system

a sulfite reservoir,

an aliquating device/driver, where the driver includes a plurality of members which may be selectively energized by the computer for the administration of sodium nitrite; and

a microprocessor to control sensors and chemical delivery.

The invention further provides a mounting apparatus for securing the system to the barrel, to prevent dislodgement during barrel movement. The invention includes a quick release mechanism for removal of the monitoring system from the barrel.

The invention further provides for an actively controlled venting apparatus in the bung. The apparatus is comprised of an aperture, a valve, a pressure sensor and a microprocessor.

The diagnostics in the bung can be divided into two groups.

The first includes optical and electrochemical measurements of, e.g., spectra, PH and temperature. These measurements are continuous and noninvasive. Data can be continuously transmitted to a central computer via wireless communications units.

The second group includes the physical extraction of samples of wine and vapors from the barrel. They can be analyzed directly in bung related analyses or removed from time to time to a central lab for detailed studies.

The collection of data from both testing groups will be maintained in a database. Evaluation of the database—comparing variables throughout the system—will allow for data mining to establish iteratively, improved winemaking algorithms.

### BRIEF DESCRIPTION OF THE DRAWINGS

The accompanying drawings, which are incorporated into and form part of this disclosure, illustrate embodiments of the invention and together with the description, serve to explain the principles of the invention.

FIGS. 1A-1C are schematic illustrations of the apparatus and method of the invention.

FIG. 2 is a schematic view of the hood part of the apparatus containing the bung and platform.

FIG. 3 is a schematic view of the stirring device and mechanism.

FIG. 4 is a schematic view of the stirring rod sensor and sampling system.

FIGS. 5A-5C are schematic views of the stirring rod based sensor.

FIG. 6 is a schematic of the laboratory unit.

### DETAILED DESCRIPTION OF THE INVENTION

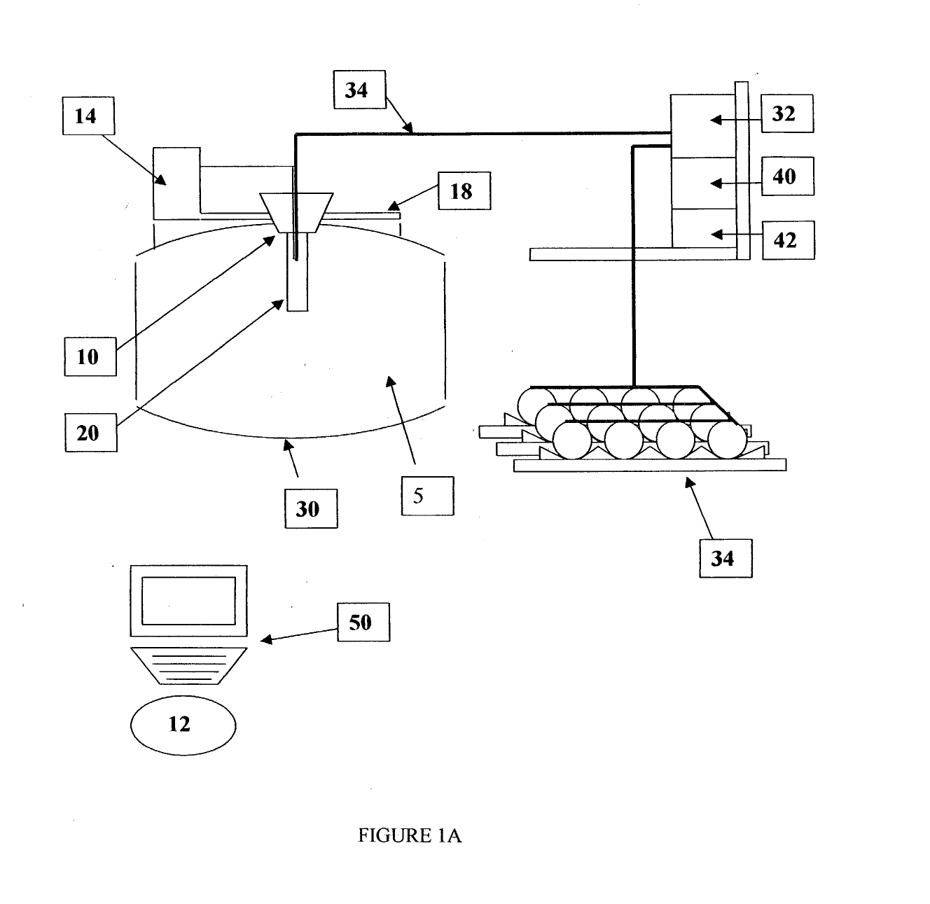

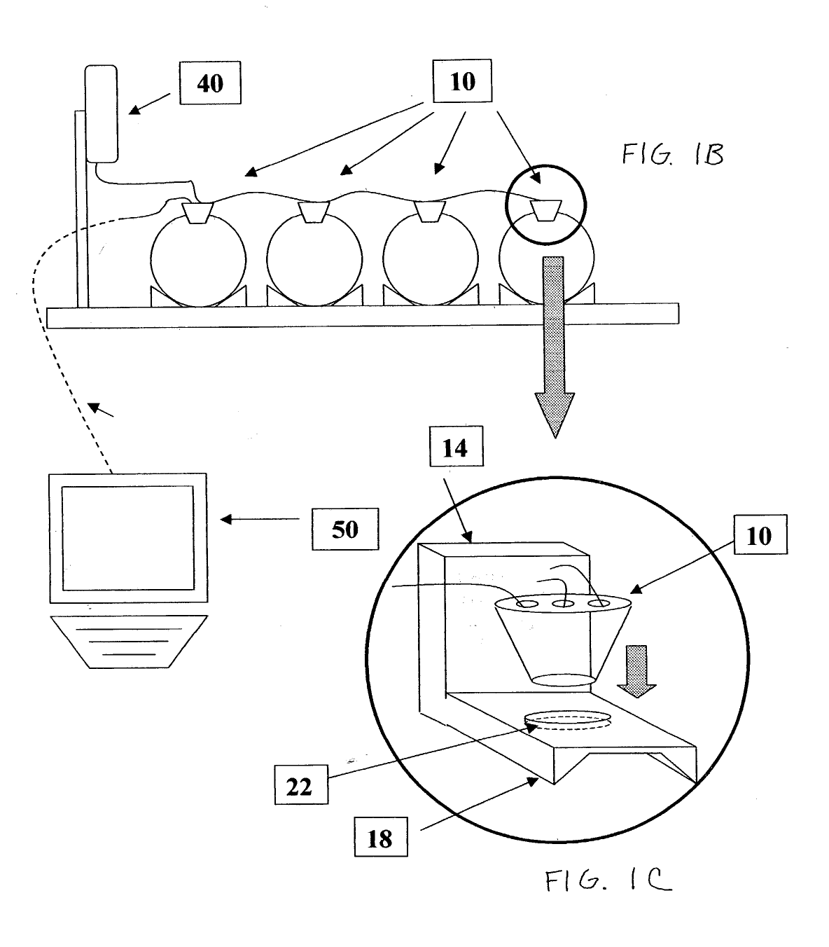

FIG. 1A illustrates how an embodiment of the present invention can be used to measure the state of wine in the barrel, stir the barrel, monitor fluid levels, deliver juice and deliver chemicals. A modular, minimally invasive wine monitoring, sensing and treatment apparatus employs a series of sensors in the bung 10, stirring rod 20, a bung related laboratory 14 and a remote laboratory 40, that can map out key characteristics of wine and barrels, and generate a performance profile of the barrels and wine throughout the production facility. The subject-user 12 has many choices in remotely monitoring and controlling the wine properties of numerous barrels, minimally invasively, without need for movement of the barrels, through a central computer in association with the barrel related system.

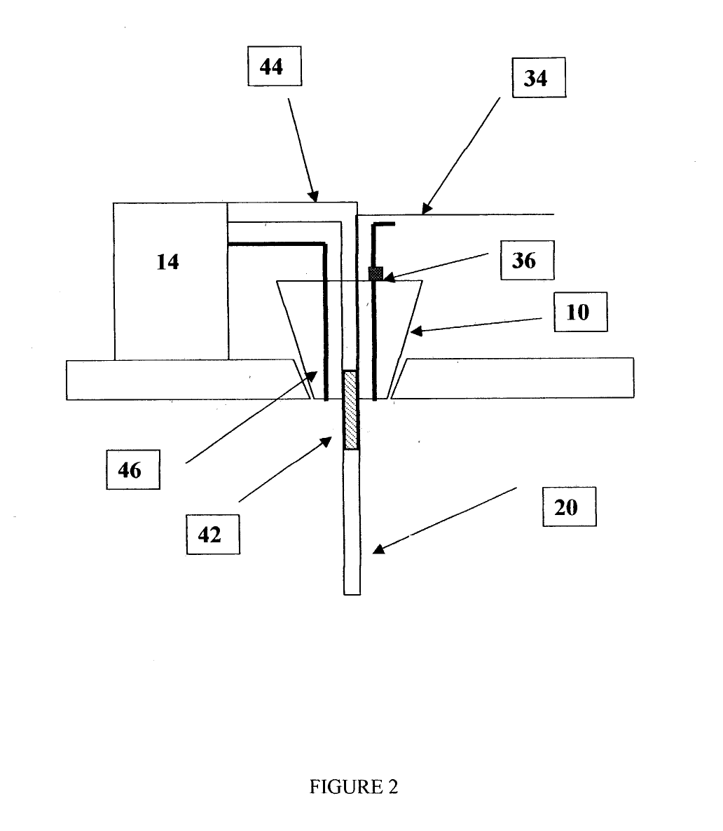

As illustrated in FIGS. 1A and 1B, a winemaker-user 12 secures the hooded system 18 to the barrel 30. The bung 10 is secured in an aperture 22 in the hooded system 18. Apertures in the bung pass conduits into the specialized stirring rod 20 from the bung laboratory unit 14 (shown in FIG. 1C and the remote laboratory 40. Via the central computer 50, the winemaker user receives and sends, electronically or telemetrically, information from the remote lab or bung units. The user, via the central computer control software, monitors physical, chemical and optical characteristics of the wine 5, monitors fluid levels, vents the wine via an active device 36 (see FIG. 2) in the bung 10, replenishes juice in the barrel from a remote reservoir 32 sent via conduit 34 that also contains tubing for gas and liquid transport to the remote lab 40. Fluid aliquot samples may be sent via tubing to remote packaging facility 42 for samples to be sent for offsite laboratory testing. Signals from the central computer control sampling and treatment from all remote and bung related modules. A module in the bung laboratory delivers chemical to the wine. The central computer analyzes and compares data values from numerous barrels of interest 38.

FIG. 2 shows an embodiment of the hood apparatus. The bung laboratory 14 receives and transmits data and instructions from central computer 12, directing control of stirring motor 42, and sampling of fluid and gas via conduits 44 to stirring rod 20. The laboratory monitors data of sensor elements in stirring rod 20 and performs analysis for numerous chemical, physical and optical properties including pH, volatile acidity, temperature, etc., as directed by the computer 50. Additional fluid is replenished by the reservoir 32 via conduits 34 directed through bung 10. Chemicals such as sulfites will be added via additional conduits 46 and vapor will be vented by valve mechanism 36.

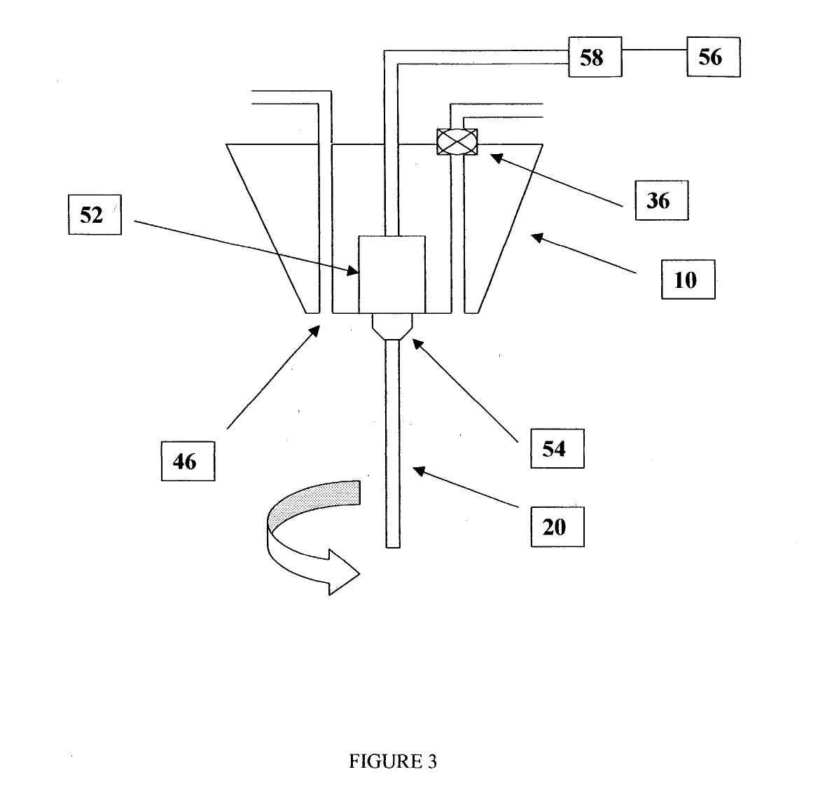

FIG. 3 is an embodiment of the stirring mechanism. Based on optical scattering data acquired by optical sensors in the rod 20, the computer sends instructions to the microprocessor 56 in the lab 14 that directs the power supply 58 to drive the motor 52 engaging the gear assembly 54 to move the stirring rod 20 to agitate the wine 5. The feedback-controlled system varies the rate and frequency of stirring to achieve the optimal user desired suspension of sedimentation in the wine correlating with the optical scattering properties.

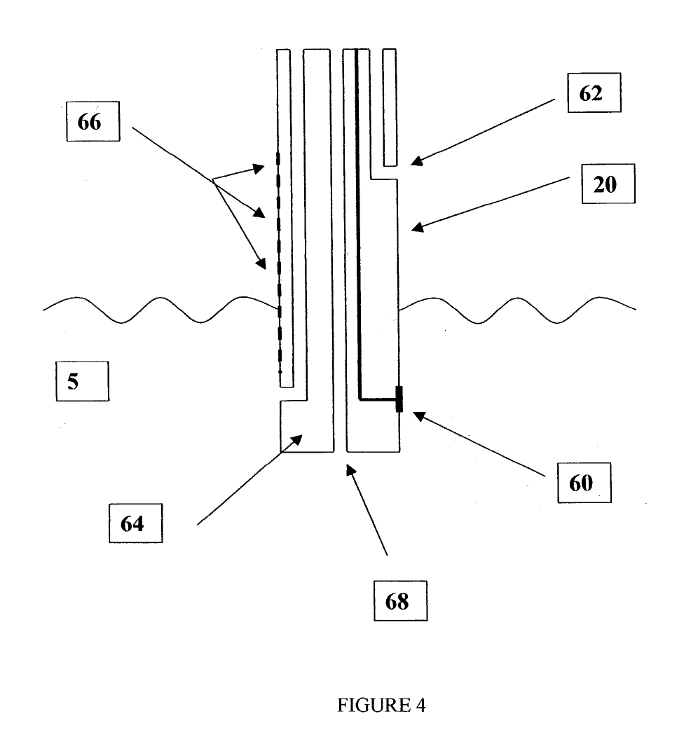

FIG. 4 shows one embodiment of the specialized stirring rod apparatus containing fluid and vapor sampling conduits and sensor elements. Based on desired sampling protocols as directed by computer 50 communicating to remote lab and bung lab microprocessors, samples of gas are aspirated from proximal apertures 62 and fluid from distal apertures 64 in the stirring rod. Electrical resistance elements 66 along the outer surface of the rod determine fluid levels to assist the user in determining evaporative losses and in deciding to replenish wine levels with juice from the reservoir 32 delivered via tubing 68. Sensor packages 60 on the rod determine optical, chemical and electrical properties of the wine.

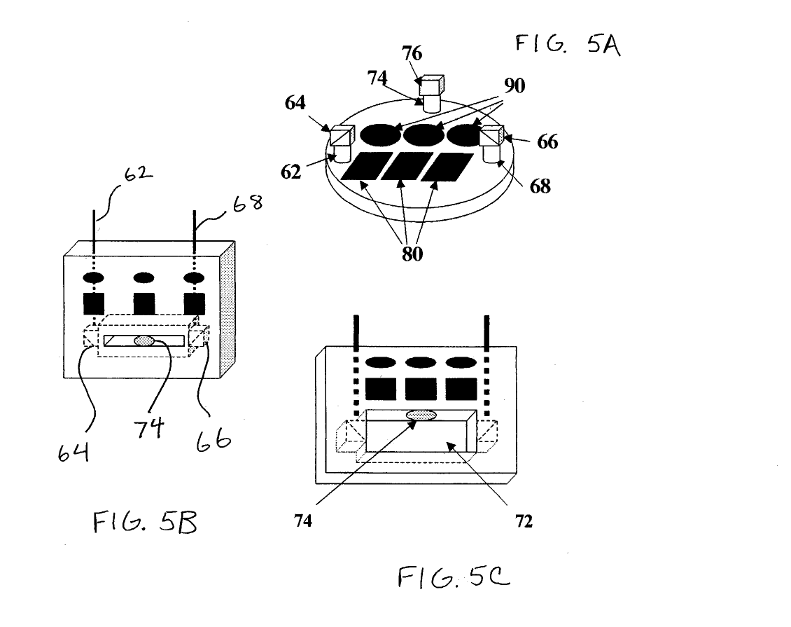

FIGS. 5A-5C show alternate views of embodiments of the sensor elements 60 that form part of the stirring rod apparatus 20 of FIG. 4. These embodiments incorporate fiber optics and electrochemical sensors to allow a wide variety of measurements. Light transmitted into the input fiber 62, by the measuring laboratory device 14 (see, e.g., FIGS. 1A-1C), is reflected by a corner cube 64 that directs the light through another corner cube 66 into a secondary fiber 68. In the barrel, wine fills the gap between the two corner cubes on the rod surface or in a chamber 72, allowing the absorption spectrum of the wine to be measured by the laboratory-measuring device 14. In order to improve light coupling between the two fibers, GRIN or other miniature lenses can be added to each fiber optic. An additional fiber 74 collects scattered light in the chamber or off corner cube 76. The measurement of scattered light can be used to determine the presence of solid material in the wine.

Electrochemical sensors 80 or optical chemical sensors 90 placed between the fibers are used to measure additional wine properties (e.g., pH, alcohol, temperature, dissolved oxygen). Liquid phase chemical sensors generally use enzymatic layers that are extremely selective to a given substrate and highly effective in increasing the rate of reactions. The enzyme is generally immobilized inside a layer into which the substrate diffuses. A wide variety of liquid phase chemical sensors currently exist which can be employed in the present invention (See e.g., “Handbook of Modern Sensors”, by Jacob Fraden 1996, and “Handbook of BioSensors and Electronic Noses Medicine, Food and the Environment”, ed Erika Kress-Rogers, 1996, incorporated herein by reference). Electrochemical sensors produce a voltage or current that is proportional to a measured quantity. Optical sensors use changes in absorption or fluorescence to measure molecular concentration or fluid property. Sensors may be placed on the rod or in a chamber within the laboratory, exposed to aliquats of wine at varied testing intervals.

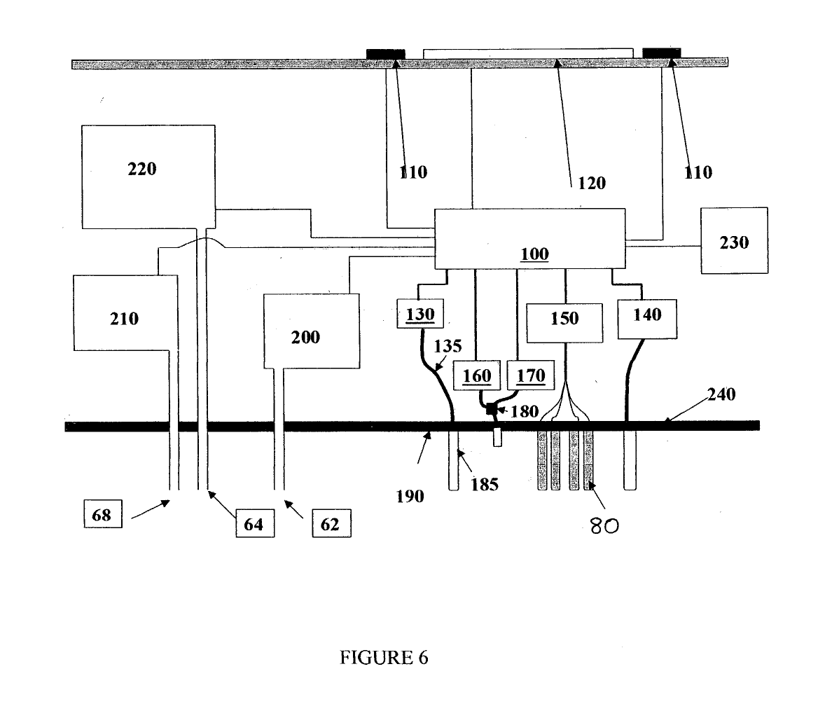

FIG. 6 shows a schematic illustration of an embodiment of the laboratory-measuring device 14. The device comprises a housing 230 that fits around the sensor package to insure proper contact for the duration of the measurement. The fiber optic 185 and electrical connection pins 195 protrude beyond the base plate 190 to dock with the sensor elements on the rod sensor package. Within the battery powered measuring device, software controls the operation of a microprocessor 100. The microprocessor receives inputs from the central computer via the transmitter/receiver 240 or locally from the user through the buttons 110. Data obtained from all sensors and modules is transmitted to the central computer via the transmitter/receiver 240. The transmitter/receiver can be wireless. Locally, the microprocessor menus and results are displayed on LCD display 120. When activated, the microprocessor 100 reads the digitized signal from all sensors. The microprocessor 100 can include an integrated analog to digital converter or require a separate analog to digital converter IC. The collected readings are analyzed by the software on the central computer and may also be displayed on the LCD display 120. In addition, for single property measurements, the measured absorption spectrum can also be displayed on the LCD display 120 as a graph.

A broad wavelength light source 130 within the device transmits light down an optical fiber 135 that goes into the sensor package and is transmitted through the wine and collected back into optical detector 140. Optical detector 140 can be a linear CCD coupled to a grating spectrometer to enable the fluorescence and/or absorption spectrum of the wine to be measured over a wavelength region extending from 300-1300 nm. Optical detector 140 could also be a set of individual filtered optical diodes that would measure the fluorescence and/or absorption characteristics at a few discreet wavelengths. The standard method of measurement is to calculate a ratio of optical density measurements at 520 nm and 420 nm. A review of the characteristic changes in the overall spectrum suggests that a scan from 350 to 600 nm contain information relative to the maturation of the wine. Another optical detector within the device measures the light collected by fiber 74 (FIG. 5).

An excitation light source 160 couples light into a fiber optic splitter 180 that sends light down to the optional fiber optic chemical sensors 90 (FIG. 5). The resulting fluorescence signal returns from the sensor package and is detected by an optical detector 170. Sensor electronics 150 power the electrochemical sensors 80 (FIG. 5) and condition the signal for the analog to digital converter. The number of sensors is only constrained by the size and target cost of the device. In addition, the type of sensor is constrained by the requirement that little or no contamination of the wine occurs.

In the preferred embodiment, all the sensor data measured by the device can be downloaded into a computer where it can be stored for future comparison. The computer can also be used to download information from the winery about interpretation of the sensor readings for varied sub groups, e.g., types of wine, barrels batches and previous years wines. In order to improve interpretation of sensor data and eliminate effects of sensor drift, each winery can maintain a group of control bottles or barrels. The data can be compared in the microprocessor to previous reading of, e.g., the same barrel, barrels at other locations, levels and previous years. The results are used to optimize winemaking algorithms. With time the winemaker will also develop a library of what sensor readings optimized for their desired winemaking preferences.

The microprocessor 100 controls the function of the MEMS laboratory 220 that receives fluid samples of wine via conduit 64 for analysis of chemical variables including sulfite concentration, pH and volatile acidity, ammonia/nitrogen content, hydrogen sulfide, thiols, amino acids and volatile acidity/acetic acid content The microchip assembly diagnostic system will also screen for molecular, antigenic and proteonomic compounds suggesting the presence of deleterious organisms or pathologic processes. The microprocessor 100 also sends commands to the chemical delivery apparatus 210 that delivers sulfites and other chemicals via conduit 68 to the barrel wine. An olfactory sensor package 200 as directed by the microprocessor receives sampling of the wine vapor via conduit 62. The vapor flows over the polymer sensors of the olfactory module 200. Data from the module is evaluated for the presence of deleterious aromatic compounds.

The computer controller 50 will receive all data and will control all operations (either electronically or telemetrically) for barrels attached to the system. Suitable computer hardware software that can be used for control of the sampling is available from LabView. Subprograms will allow for review of the data and optimization of sampling and wine-making algorithms based on the additional and more extensive data and information of the physical, chemical and biological behavior of the wine that this system provides. A central winemaking database may be established for data mining by winemakers or desired parameter profiles for different winemaking and wine types throughout the production process.

If the presence of nondesirable aromatic compounds is suspected and more extensive testing is desired beyond the capabilities of the bung system, the computer will direct the remote packaging module 42 to remove an aliquot of the fluid and seal it in a container for shipping to an off site laboratory. The central computer will also control the remote laboratory 40 and reservoir system 32. The remote lab may include one of an array of gas chromatography or mass spectroscopy technologies for assessing low concentration compounds and elements within the wine of any barrel attached to the system.

Although the invention has been described with reference to certain preferred embodiments, it will be appreciated that many variations and modifications may be made within the scope of the broad principles of the invention. For example, this invention can be used for any drinking fluid or liquid that requires a prolonged barrel aging process. Hence, it is intended that the preferred embodiments and all of such variations and modifications be included within the scope and spirit of the invention.

We claim:

1. An apparatus for adjusting the contents of a fermenting liquid, comprising;
a stopper configured for placement in an opening of a container used for containing a fermenting liquid;
at least one sensor element operably connected to said stopper to sense a parameter of said fermenting liquid; and
means for altering the contents of said fermenting liquid to change said parameter.

2. The apparatus of claim 1, wherein said stopper comprises a bung, wherein said container comprises a cask and wherein said fermenting liquid comprises wine.

3. The apparatus of claim 1, wherein said at least one sensor element is selected from the group consisting of a chemical sensor element, a physical sensor element and an optical sensor element.

4. The apparatus of claim 3, wherein said chemical sensor element is configured to measure a parameter selected from the group consisting of pH, volatile acidity, alcohol, dissolved oxygen, sulfite concentration, ammonia/nitrogen content, hydrogen sulfide, a thiol, an amino acid, a molecular compound, an antigenic compound and a proteonomic compound.

5. The apparatus of claim 3, wherein said chemical sensor element is selected from the group consisting of an electrochemical sensor element, a liquid phase chemical sensor element, a MEMS laboratory, a polymer sensor element and a gas chromatograph.

6. The apparatus of claim 3, wherein said physical sensor element is selected from the group consisting of a temperature sensor element, an electrical resistance sensor element and a mass spectrometer.

7. The apparatus of claim 3, wherein said optical sensor element is selected from the group consisting of a photodetector element, a fiber optic and a fluorescence detector.

8. The apparatus of claim 1, wherein said means for altering the contents of said fermenting liquid to change said parameter comprises a computer with software that reads said parameter, compares said parameter with a desired parameter and controls a mechanism that alters the contents of said fermenting liquid to change said parameter.

9. A method for adjusting the contents of a fermenting liquid, comprising;
sensing a parameter of a fermenting liquid with at least one sensor element operably connected to a stopper placed in an opening of a container containing said fermenting liquid; and
altering the contents of said fermenting liquid to change said parameter.

10. An apparatus for adjusting the contents of a fermenting liquid, comprising;
at least one sensor element operably connected to a container, wherein said at least one sensor element is configured to measure a parameter of a fermenting liquid in said container; and
means for communicating said parameter from said at least sensor element to another location.

11. The apparatus of claim 10, wherein said means for communicating comprise a radio frequency transmitter.

12. The apparatus of claim 10, wherein said means for communicating comprise a wireless transmitter.
